(19) United States
(12) Patent Application Publication
(10) Pub. No.: US 2026/0026695 A1
(43) Pub. Date: Jan. 29, 2026

(54) MONITORING OF ENDOLEAKS

(71) Applicant: DEERFIELD CATALYST, LLC, New York, NY (US)

(72) Inventor: Mark GELFAND, New York, NY (US)

(21) Appl. No.: 19/100,863

(22) PCT Filed: Aug. 3, 2023
(86) PCT No.: PCT/US2023/071607

§ 371 Date: Feb. 3, 2025

Related U.S. Application Data
(60) Provisional application No. 63/394,993, filed on Aug. 4, 2022.

(51) Int. Cl. A61B 5/02; A61B 5/00; A61B 5/05; H04B 5/70

(52) U.S. Cl. CPC A61B 5/02014; A61B 5/05; A61B 5/6846; H04B 5/70

(57) ABSTRACT

A system including: an antenna configured to be implanted in a region a living body, wherein a frequency response and/or attenuation response of the antenna shifts in response to changes in a physiological condition of the living body at the region; an input/output module outside the living body and configured to actuate the antenna to generate a response signal and to receive the response signal, wherein the response signal indicates a frequency response and/or attenuation response of the antenna; and a processor configured to: detect whether the frequency response and/or attenuation response indicates a shift in the frequency and/or attenuation response of the antenna; and in response to the detection of the shift issue and/or record a notice that a change occurred in the physiological condition at the region inside the living body.

### FIELD OF INVENTION

The present invention relates to a system and method for monitoring endoleaks in an aneurism sac after the implanting of a stent graft in an artery.

### BACKGROUND

Endovascular aneurysm repair (EVAR) is a viable, less invasive alternative to the highly invasive open repair for abdominal aortic aneurysms. Endovascular repair of abdominal aortic aneurysms is dependent on the successful exclusion of the aneurysm from arterial circulation. Unfortunately, endoleaks are a complication unique to EVAR and can occur in up to 25% of patients.

A common type of EVAR graft is an endovascular stent graft used to treat an abdominal aortic aneurism. The endovascular stent graft is placed inside the aneurysm and acts as an artificial lumen through which blood can travel, instead of flowing into the aneurysm sac, and is designed to help prevent an aneurism from bursting. The sac is therefore excluded from blood circulation. Examples of EVAR stent graft devices are Zenith (Cook, Indianapolis, Ind), Excluder (WL Gore, Flagstaff, Ariz;), AneuRx (Medtronic, Santa Rosa, Calif) and others. Regardless of the device used, to perform endovascular stent graft implantations, a surgeon will insert the stent graft into the blood vessel at the location of the aneurism using an arterial catheter delivery system in order to reduce the pressure on the blood vessel walls at the site of the aneurism. Stent grafts and delivery systems have been used widely for many years and are well known. Unfortunately, EVAR stent grafts are sometimes subject to failure.

One type of failure that may occur is the leaking of blood into the aneurysm sac; a condition referred to as an endoleak, of which there are 5 different types.

A Type I Endoleak occurs when blood flows between the stent graft and the blood vessel wall; typically at the proximal (often renal) or distal (often iliac) end of the graft. This complication may also occur as a result of movement of the graft away from the desired location, sometimes called migration.

Type II Endoleaks occur when blood flows backwards (retrograde) into the aneurysm sac from collateral arteries originating from the aneurysm sac itself (typically the lumbar, testicular or inferior mesenteric arteries).

Type III Endoleaks occur when blood leaks between the junction sites of “articulated” or “segmented” stent grafts; these multi-component stent grafts are inserted as separate segments which are then assembled inside the artery into their final configuration. Detecting and confirming accurate assembly and fluid-tight contact between the different segments is difficult and current verification methods of correct assembly are suboptimal.

Type IV Endoleaks occur when cracks or defects develop in the stent graft fabric and blood is able to leak directly through the graft material. This may also occur due to the natural porosity of the graft material (polyester, PET).

Type V Endoleaks are the leakage of blood into the aneurysm sac of an unknown origin. Regardless of their cause, endoleaks are frequently a medical emergency and early detection, characterization and post-operative and long-term monitoring of them is an important unmet medical need.

### SUMMARY

Several methods and devices are proposed for detection of endoleaks. Some of these methods are based on the notion that physical properties, such as pulsatile motion, acoustic permeability, viscoelastic properties, and dielectric impedance of the aneurism sack, will change in a relatively predictable manner if an endoleak develops and that these changes can be detected by less expensive and more robust means.

One aspect of the technology utilizes RFID (radio frequency identification) technology. A possible application of the proposed sensing paradigm may be the monitoring of endoleaks via monitoring of blood flow. By acquisition of the sensors' response at different times (days or even hours) a map or a 3D model of the geometrical or chemical changes of the aneurysm sac and stent graft interface may be produced, thus evaluating, and possibly avoiding possible complications and intervening in time to prevent severe outcomes.

Accelerometry is a reliable estimate of pulsatile motion that can be related to pulse pressure (index of leaks). For example, it is proposed to measure the acceleration of the aortic wall, thrombus and endograft in targeted locations. An integral of the acceleration after the removal of drift with a high pass filter can be another reliable indicator of a physiologic trend. Implanted accelerometers are not subject to interference from biologic media and can be completely encapsulated and insulated from chemical reaction with blood and body fluids. They are extremely small and require very low power. Since accelerometers are relatively inexpensive and require low power, multiple devices can be implanted in one system. One accelerometer can be implanted on the graft itself and one on the aortic wall inside the sack and yet another one on the aortic wall outside of the graft. In one embodiment, a trend in the difference between the motion profile of the graft wall and the aortic wall is used as an indication of an endoleak.

In addition to simple amplitude of pulsations, the pulse wave shape can be interrogated to determine endoleaks. Arterial pulse wave profile can be a predictor of vascular resistance and impedance in the setting of vasoconstriction. It can be anticipated that pressure pulses transmitted by the aneurism sac to the aortic wall can get steeper or shallower depending on the amount of endoleak. This can result in the changes to the acceleration and velocity of the aortic wall itself. Type II endoleaks may be detected by analyzing the time it takes for the pulse to arrive at the sensor. For example, a delay from the surface EKG detection can indicate the circulatory delay.

It is contemplated that the device may include an electronics capsule, also deployable through a catheter, having an ultrasound transducer, or an electric impedance monitoring circuit containing at least power and communications modules controlled by an external console device to receive and wirelessly transmit signals based on inputs from the ultrasound or impedance transducer.

A small ultrasound device (home or doctor's office based) can detect changes in some key factors (for example the diameter of the sac, the distance between landmarks on the sac, the distance between sac and adjacent body structures) and alert the user of the need for a more detailed scan. Additional echogenic markers can be added to the stent graft material and implanted inside the sac, possibly attached to the wall of the aneurism sac to target the ultrasound beam and simplify calculations. Echogenic markers may comprise a simple echo-reflective tube such as a sealed tube of air, a coating that traps gas bubbles, or a metal surface with specific etching. Markers may have specific geometry, such as lines and crosses, easily detectable and recognizable by the software imbedded in the external monitoring device.

The Doppler ultrasound velocities of type II endoleaks can predict the progression of type II endoleaks and increased sac growth. In particular, ultrasound is attenuated when it travels through a medium depending on the properties of the medium.

Water and blood have very low acoustic resistance, and ultrasound waves travel with high speed, whereas air and bony structures are almost impenetrable and reflect the ultrasonographic waves almost completely. The speed of sound is 1,540 m/sec in average water containing tissue at 37° C. Differences from this standard can be an index of clot uniformity.

Blood velocity can be measured using pulse Doppler. The result of the processing is the difference in frequency (Doppler shift) between the master oscillator and the signals reflected from the flow sensitive region as defined by the adjustable receiver gate. The relationship between the Doppler shift and absolute velocity can be exploited to detect blood moving inside the aneurism sac.

The movement of blood generates a specific sound signature in the acoustic range that is reflected as vibrations detectable by piezoelectric sensors that can serve as both emitters and receivers. These sensors can be miniaturized and implanted as part of the aneurism leak monitoring system.

In other embodiments, electric impedance of body tissues and fluids (also called bioimpedance), which may comprise resistance, inductance and capacitance, and their change over time can also be measured to identify endoleaks. There are several ways in which the impedance of the aneurism sac can be altered by endoleaks. For example, impedance (electric resistance and capacitance) of a blood clot is different from the impedance of blood.

Impedance measurement or “bioimpedance” is a complex variable that reflects resistance and capacitance of tissues in the path of alternating current. It is a function of the excitation frequency at which impedance is measured. Measurements of impedance across several frequencies or frequency ranges are called impedance spectrum or impedance spectroscopy. Resistivity is a measure of resistance frequently used in relevant literature and adjusted by the electrode calibration constant to correct for small physical differences between electrodes.

Both individual cells and biologic tissue can be ideally modeled with a simple equivalent circuit in which extracellular resistance (Re), intracellular fluid resistance (Ri), and cell membrane capacitance (Cm) are represented. Individual real time measurements of electric current and voltage allow determination of resistance and capacitance at different frequencies. Such circuits and algorithms are well described in literature and term “impedance” used here incudes such measurements.

Impedance (Z) is a measure of the total opposition to current flow in an alternating current (AC) circuit and is defined in terms of its three individual components: resistance (R), inductance (L), and capacitance (C).

$$z = R + j ω L + \frac{1}{j ω C}$$

where ω=angular frequency

The angular frequency of the current is represented by omega, and j is the square root of (−1). Impedance can best be understood as the AC correlate of resistance (R) in direct current (DC) circuits (R=V/I), and is likewise expressed in Ohms. Body tissues and physiological fluids have known characteristic ranges of impedance values that may vary in individuals and require a baseline calibration. There can be distinguished, an examination on a whole body scale, as fat-free mass, total body water, intracellular water, extracellular water and body cell mass or the body bigger parts (e.g., rheography and plethysmography), examination of particular organs, glands or parts of the body (e.g., heart, liver, larynx, prostate, breast, blood, etc., examination of some selected fragments (e.g., skin changes in dermatology), and also examinations performed on a small scale (e.g., impedance measurements of a single cell).

Impedance of blood and dense tissues such as muscle, fibrous tissue, fat and bone may be lower than the impedance of the blood clot in general. However, there are challenges in measuring localized impedances post EVAR when the stent-aneurysm interface can have impedance variability in response to the injury, which might also show impedance fluctuations which can get incorrectly classified as blood flow related impedance changes pertinent to endoleaks.

The low frequency movement of the smooth muscle cells and the fibrotic tissue growth may be indistinguishable from blood-flow related changes in the resistive component of impedance, however as the frequency of impedance measurement increases, it would be possible to see the difference between the capacitive component of impedance, specifically in the 10-100 KHz range.

In one embodiment a normal healing trend is anticipated where day to day or hour to hour impedance is expected to increase as the healing and clotting of the sac occurs. Remote analytics can periodically update the trend and display it to the physician as needed. If the trend reverses and does not revert back in a predetermined time, it may be an alarming sign indicating that a persistent endoleak has developed.

Electric impedance of blood is much lower than the bioimpedance of a blood clot when measured in the range of frequency between 500 and 20,000 Hz and specifically 5,000 and 10,000 Hz. Monitoring of bioimpedance does not involve moving parts or sensors that deteriorate with time. It can be measured quickly, for example, in a time period of 5-10 milliseconds. It is advantageous since it allows low electric energy consumption and conservation of battery life.

Blood leakage may act as an electric shunt since liquid blood has the lowest dielectric resistance to ionic conduction among all body tissues and structures. Local capacitance of the thrombus material may also be significantly altered by blood leaks when measured at a higher frequency of AC current.

Alternatively, as the aneurysm sac fills, its walls will stretch away from the stent graft walls. It is possible to think of the sac space as a capacitor since the farther away the stent graft wall goes away from the sac walls its capacitance will decrease.

In the real world setting where blood leaks may only partially fill the sack, the impedance trend may be more important than the absolute number. Impedance may be monitored for the individual patient over hours, days, months, and years after surgery. It is expected that initially, impedance will increase gradually as the clot forms and then reach a plateau. That plateau may be registered as the patient's baseline impedance. If the patient develops an endoleak, impedance may suddenly or gradually decrease indicating a good reason for a check using CT or other image-based monitoring means.

In general, the volume sensitivity of an impedance measurement will be a function of the square of the current density in a given tissue volume. Unipolar, bipolar, and multipolar measurements can be applied. A unipolar measurement may have an electrode setup resulting in a high current density adjacent to the active electrode surface compared to the rest of the tissue. In this way, the measured impedance will be dominated by the vicinity of the active electrode, and dependent on the area and geometry of the electrode.

The impedance data will reflect averaging over the tissue in the dominant sensitivity volume. The smaller the active electrode area, the higher the obtained spatial resolution. For example, for a hemispheric electrode in a homogenous medium, 90% of the measured resistance is expected to be due to the volume within a radius ten times the radius of the electrode.

For example, if the electrode may be a hemisphere with a diameter of 2 mm impedance will be measured in the radius of approximately 10 mm of a surrounding sensitivity zone. Localized impedance measurement may be employed at the specific points where endoleaks are likely such as at the proximal and distal ends, at the joints of the joined multi-section graft or where small arteries branch from the aorta inside the sac and retrograde perfusion is possible.

Alternatively, the bipolar impedance can be measured between the electrodes at a distance along the endograft sac. In this case there is no dominant electrode since the surface area of electrodes is similar and the measurement is localized by the virtue of the current return path and reflects the average impedance of the clot and blood in the sack. For example, anterior, posterior, and lateral current paths can be included in the design.

In one configuration, an implantable device is capable of generating a carrier waveform that is passed though the targeted local tissue volume and the impedance of the current pathway or the volume of interest is analyzed. Impedance may be measured periodically and automatically with the general goal of detecting endoleaks. Endoleaks may be detected as a drop or a persistent declining trend in impedance compared to baseline values or compared to different reference locations in the aneurism sac and may be generally characterized as shunting of current and may include changes of resistance and capacitance.

At least one impedance sensing electrode may be an approximately 2 mm metal sphere or hemisphere electrode or approximate a sphere. Alternatively, the impedance sensing electrode can be a ring electrode. The sensitivity zone is a space defined by the distance of approximately 10 mm. Impedance measurements will be approximately defined by the average impedance of tissue within the tissue sphere. Local impedance of blood is expected to be lower than the impedance of the blood clot.

Alternatively, the bipolar or tripolar impedance can be measured between the electrodes at a distance along the endograft sac (See FIGS. 3 and 5). In this case there is no dominant electrode since the surface area of electrodes is similar and the measurement is localized by the virtue of the current return path and reflects the average impedance of the clot and blood in the sack along that path. For example, anterior, posterior, and lateral current paths can be included in the design reflecting the orientation of the graft relative to the body of the patient.

Some configurations include an implantable device capable of generating a carrier waveform, passing the carrier waveform through the targeted local tissue volume, and analyzing the impedance of the current pathway or the volume of interest. Impedance may be measured periodically and automatically with the general goal of detecting endoleaks. Endoleaks may be detected as a drop of impedance compared to historic values or the impedance difference between different reference locations in the aneurism sac and may be generally characterized as shunting of current and may include changes of resistance and capacitance.

Implantable devices that require power to operate may be battery operated (active) or wireless (passive). Passive, wireless devices powered by external devices when used are generally preferred due to their simplicity and lower cost.

In some embodiments the device comprises passive or active marker elements coupled to a deployable expandable structure for deployment into the aneurism sac in a collapsed state via a catheter. The device may comprise anchor elements to secure and immobilize the device in the sac. The structure may be expandable between a first, collapsed configuration and a second, deployed expanded configuration, wherein the first collapsed configuration has an overall diameter sufficient to fit in the catheter, such as 14 F (French) to 17 F, and the second expanded deployed condition has an overall diameter sufficient to securely engage the aortic wall lumen at the anchoring location and in some embodiments, create a current path. In yet other embodiments, the structure may be expandable to create an RF antenna. A deployable structure may be configured to coil around the perimeter of the sac at least partially enveloping the stent graft whilst the markers or sensors are positioned along the length of the device.

Battery operated implantable devices can be put into a sleep mode, where essentially only clock circuitry is powered until certain time of the day when they wake up and perform measurements that can be stored in memory. Alternatively, devices may be activated as needed by an external wireless RF communication signal. Once a day or at any other suitable time interval the implantable device can be interrogated by telemetry and collected data can be sent to the physician. To avoid errors, impedance measurements collected during the day can be filtered and averaged. In vitro impedance of whole blood may be on the order of 100 to 1,000 ohms while impedance of blood clot may be on the order of 0.1-1.0 megaohms. Impedance of the mixture filling the sac may be expected to fit in this broad range and depend on the amount of liquid blood.

One embodiment to achieve the ability to reliably detect endoleaks (as opposed to a wound response, for example) is via use of an EVAR stent graft modified by the addition of multiple acceleration or bioimpedance sensors. The stent graft will have multiple sensors incorporated throughout it to detect simultaneous impedance changes due to wound response related impedance change. This may also be expanded further to detect cross-sectional reduction of size, pressure, and/or flow. The endoleak can be detected when there is at least one or more impedance sensor that shows an anomaly or change of normal healing trend compared to another impedance sensor. Each “sensing” unit may be an integrated microcircuit consisting of a sensor, conditioning circuitry for control of a transceiver and impedance analyzer tuned to a said frequency in order to reduce PCB footprint, an antenna, and a power unit which could be energized through wireless power to reduce or eliminate the need for a battery in the implant. This data can be relayed through the cloud through secure connections allowing an automated AI processor or a doctor to securely log into a patient data to remotely assess the patient and provide treatment updates if necessary.

To report the impedance from the sensor-stent a miniaturized telemetry device based on an AD5933 (Analog Device) can be developed. A triple layer PCB of 3.5 by 1 cm can be made and encapsulated in biocompatible acrylic. The sensor assembly can be integrated into a custom nitinol stent. Nitinol is a corrosion resistant and memory alloy that is ideal for the placement of the sensor on the stent and that is capable of being radially expanded without deformation in patients with EVAR. The non-deformable platform may measure approximately 2×5 mm with a “sensing” window of 2.1×1 mm. The stent pattern and diameter may be generic (for example, 10 mm with a length of 20 mm) and custom designed to accommodate our current sensor. The sensor island or window allows for the orientation of the sensing units to be facing toward the luminal side of the artery, but could equally be reversed for abluminal or wall detection, while the excess bulk of the sensor is clear of the lumen and may be embedded within the stent structure. Bonding of the sensor to this type of stent means the sensor island is less prone to damage and as such failure of the sensor to the stent is less likely.

In one configuration, a stent antenna for intravascular monitoring and implantable wireless applications is proposed. In this embodiment the entire stent is an antenna. The stent antenna may have better radiation performance than that of LC resonant antennas at low frequencies, such as 900 MHz. Modern stents are composed of open cells, each unit is composed of crowns and struts, and each ring is connected by multiple connectors. The proposed design of a stent antenna with good radiation performance may be achieved by using a single connector between each unit of the stent. The single-connector stent antenna eliminates the complicated and meandering geometry of a typical stent, which would cancel out the induced currents and be unable to excite the desired electromagnetic waves. In addition, a stent with single connectors should maintain a stable mechanical structure and not exceed fracture limits during balloon expansion. The single-connector stent antenna may be applied to other vessels and not limited to coronary arteries.

Another aspect of the technology is a method comprising: monitoring a frequency response of an antenna implanted within a region of a living body of a mammal that can be a human patient, where the antenna may be an RFID tag, wherein the frequency response of the antenna is sensitive to a physiological condition at the region; detecting a shift in the frequency response during the step of monitoring; and in response to the detection of the shift, issuing and recording a notice that a change occurred in the physiological condition at the region the living body.

The method may include, transmitting an electromagnetic signal into the living body, followed by the step of monitoring the antenna response to the transmission of the electromagnetic signal. The step of monitoring the frequency response may include monitoring a resonant frequency of the antenna and attenuation of the signal by the conduction media.

The region of the living body is a surgical site or a wound, such as a surgical incision or wound. The physiological condition may be bleeding at the region. The region may contain a foreign body implant. The condition may include secretion of fluid such as blood, transudate or exudate from blood capillaries and interstitial edema.

The antenna may be a stent antenna, and the region is in a vascular system of the living body.

The step of monitoring may be performed while the living body heals in the region.

The antenna may be passive and not wired to a source of electric or electromagnetic energy. The antenna may be included in a radio-frequency identification (RFID) device implanted in the living body. The antenna may be a meandered antenna. The antenna may be attached or printed on a dielectric substrate and enclosed between dielectric layers.

The antenna may be included in an orthopedic implant or a breast implant and the region is a breast into which the breast has been implanted. The physiological condition indicates a malfunction of the breast implant. The malfunction may be a leakage of the breast implant into the breast implant capsule which is expected to change the response characteristic of the RFID antenna. In some embodiments the implanted RFID can also perform typical data storage and communication functions such as encoded identification of the patient or an implant. A wireless RFID circuit may be part of the stent graft or a separate “drop in” that does not require any battery but performs either a FSK (Frequency shift keying) or ASK (Amplitude Shift keying) function.

Another aspect of the technology includes a method for monitoring a physiological condition inside a living body, the method comprising: monitoring a frequency response and/or attenuation response of an antenna implanted within a region inside the living body, wherein the frequency and/or attenuation response of the antenna shifts in response to changes in the physiological condition at the region; detecting a shift in the frequency and/or attenuation response during the step of monitoring; and in response to the detection of the shift in the frequency and/or attenuation response, issuing and/or recording a notice that a change occurred in the physiological condition at the region inside the living body.

The physiological condition may be an endoleak. The region may be an isolated aneurism sac. The physiological condition may be a process of blood clotting happening over time. The process can be naturally reversed by blood leaks into the sac region. The reverse process may accelerate, and the acceleration may be detected and indicated. The region may be the predetermined location inside the sac. The predetermined location may be near the distal seal of the EVAR graft. The predetermined location may be proximate to a collateral blood vessel terminating in the sac.

The shift in the frequency response may be a shift of a resonant frequency of the antenna. The shift in the attenuation response may be a shift in an amplitude of the attenuation response. The shift may occur in response to a healing of a wound or surgical incision at the region. The healing may occur seven to thirty days after the antenna is inserted into the region. The step of detecting the shift may include detecting a rate of change of the shift. The step of detecting the shift may include repeatedly detecting the shift, storing each of the detected shifts and a time of each detected shift.

The method may further include determining whether to issue and/or record the notice based on the detected shifts and the times. The step of determining may include determining that the shift persists for at least one of: an hour, three to five hours, and one to three days.

The method may further include transmitting an electromagnetic signal into the region inside the living body to actuate the frequency response and/or attenuation response of the antenna. The step of monitoring the frequency response may include monitoring a resonant frequency of the antenna. The region inside the living body may be a surgical site or a wound. The physiological condition may be bleeding at the region. The physiological condition may be an accumulation of body fluid in the region. The antenna may be a stent antenna, and the region is in a vascular system of the living body. The step of monitoring may be performed while the living body heals in the region. The antenna may be passive and may not be wired to a source of electromagnetic energy. The antenna may be included in a radio-frequency identification (RFID) device implanted in the living body. The RFID device may be a passive RFID device. The RFID device may include a meandered PIFA antenna.

Another aspect of the technology includes a method for monitoring a physiological condition inside an aneurism sac inside a living body, the method comprising: monitoring a frequency response and/or attenuation response of an antenna implanted within an aneurism sac excluded from circulation by a endovascular aneurysm repair stent graft, the frequency and/or attenuation response of the antenna being sensitive to the physiological condition inside the aneurism sac; detecting a shift in the frequency and/or attenuation response during the step of monitoring; and in response to the detection of the shift, issuing and/or recording a notice that a change occurred in the physiological condition.

The shift in the frequency response may be a shift of a resonance frequency of the antenna. The shift in the attenuation response may be a shift in an amplitude of the attenuation response. The step of detecting the shift may include detecting a rate of change of the shift. The step of detecting the shift may include repeatedly detecting the shift, storing each of the detected shifts and a time of each detected shift.

The method may further include determining whether to issue and/or record the notice based on the detected shifts and the times. The method may further include transmitting an electromagnetic signal into the aneurism sac to actuate the frequency response and/or attenuation response of the antenna. The physiological condition may be bleeding into the aneurism sac. The antenna may be a stent antenna. The antenna may be passive and is not wired to a source of electromagnetic energy. The antenna may be included in a radio-frequency identification device (RFID) implanted in the living body. The RFID device may be a passive RFID device. The RFID device may include a meandered PIFA antenna. The antenna may not be connected to another sensor in the living body.

Another aspect of the technology includes a method for monitoring a physiological condition at a region located inside a living body, the method comprising: actuating an antenna to generate a response signal, the antenna being implanted at the region inside the living body; receiving the response signal at a location outside of the living body; comparing a frequency and/or attenuation response of the response signal to a previously recorded reference signal to determine whether the frequency and/or attenuation response has changed; and activating an indicator and/or recording a notice when the frequency and/or attenuation response of the antenna has changed, wherein a change in the frequency and/or attenuation response of the antenna indicates that a change occurred in the physiological condition.

The region may be an aneurism sac. The physiological condition may be an amount of blood or fluid leaking into the aneurism sac. A difference between the resonant frequency of the response signal and the resonant frequency of the reference signal that is greater than a threshold may indicate a leakage of blood into an aneurism sac. A difference between the attenuation of the response signal and the attenuation of the reference signal that is greater than a threshold may indicate a leakage of blood into an aneurism sac. The antenna may be periodically actuated, and the quality of the subsequent response signal may be compared to the quality of the reference signal and/or previous response signals. The antenna may be a radio-frequency identification (RFID) device. The response signal may be received on a posterior side of the living body.

Another aspect of the technology includes a method for monitoring a physiological condition at a region located inside a living body, the method comprising: actuating an antenna to generate a reference signal from the region located inside the living body; receiving the reference signal at a location outside the living body; recording a resonant frequency and/or return loss of the reference signal after it has been received; subsequently actuating the antenna again to generate a subsequent signal from inside the living body; receiving the subsequent signal outside the living body; comparing a resonant frequency and/or a return loss of the subsequent signal to the resonant frequency and/or return loss of the reference signal; and activating an indicator and/or recording a notice when the resonant frequency and/or return loss has changed, wherein a change in the resonant frequency and/or return loss indicates that a change occurred in the physiological condition.

The region may be an aneurism sac. The physiological condition may be an amount of liquid blood leaking into the aneurism sac. A difference between the resonant frequency of the subsequent signal and the resonant frequency of the reference signal that is greater than a threshold may indicate a leakage of blood into an aneurism sac. A difference between the attenuation of the subsequent signal and the attenuation of the reference signal that is greater than a threshold may indicate a leakage of blood into an aneurism sac. The antenna may be a radio-frequency identification (RFID) device. The subsequent signal may be received on a posterior side of the living body.

Another aspect of the technology includes a system configured to monitor a physiological condition at a region located inside a living body, the system comprising: an antenna configured to be implanted at the region inside the living body; a signal detector located outside the living body and configured to actuate the antenna to generate a response signal, the signal detector being further configured to receive the response signal; and a processor configured to compare a resonant frequency and/or an attenuation of the response signal to a resonant frequency and/or an attenuation of a reference signal, wherein the processor is configured to activate an indicator and/or record a notice when the resonant frequency and/or the attenuation has changed, and wherein a change in the resonant frequency and/or the attenuation is indicative of a change in the physiological condition.

The region may be an aneurism sac. The physiological condition may be an amount of blood or fluid leaking into the aneurism sac. A difference between the resonant frequency of the response signal and the resonant frequency of the reference signal that is greater than a threshold may indicate a leakage of blood into the aneurism sac. A difference between the attenuation of the response signal and the attenuation of the reference signal that is greater than a threshold may indicate a leakage of blood into the aneurism sac. The antenna may be periodically actuated, and the characteristic of the subsequent response signal is compared to the characteristic of the reference signal and/or previous response signals. The antenna may be a radio-frequency identification (RFID) device. The signal detector may be configured to receive the response signal on a posterior side of the living body.

Another aspect of the technology includes a system for monitoring a physiological condition inside a patient's body, the system comprising: a passive implantable antenna adapted for placement in an aneurism sac of the patient during an endovascular procedure; and a transmitter and a receiver that are external to the patient's body, wherein the transmitter is configured to trigger a response signal from the passive implantable antenna, and wherein the receiver is configured to determine the presence of an endoleak in the aneurism sac based on the antenna's resonant frequency and/or the antenna's impedance.

The transmitter and the receiver may be configured to be in radio communication with the antenna. The receiver may be configured to receive a radio signal indicative of the antenna's resonant frequency and/or impedance. A shift of the resonant frequency and/or or a change in the impedance may be indicative of a penetration of liquid into the aneurism sac. The antenna may be configured so that the resonant frequency varies based on the impedance of the surrounding environment, and the variance of the resonant frequency is indicative of a difference between a clot and liquid blood. The receiver may be configured to determine the presence of an endoleak by detecting a gradual increase of signal loss encountered when communicating with an RFID antenna. The receiver may be configured to determine a catastrophic endoleak by detecting an abrupt increase in signal loss encountered when communicating with an RFID antenna.

Another aspect of the technology includes a vascular implant system comprising: an antenna configured to generate a signal with a predetermined resonant frequency in response to receiving a radio signal from a transmitter, wherein the antenna is adapted for periprocedural delivery into an aneurism sac, wherein the antenna comprises a resilient metal structure that has two ends attached to a flat substrate made of flexible dielectric polymer and is covered with a flexible polymer coating to form a flex circuit.

The procedure may be the placement of a stent graft to isolate the aneurism sac from an artery lumen. The antenna may be foldable. The antenna may be configured to be rolled or compressed into a tube that is deliverable into the aneurism sac. The flex circuit may have an electrical property with a known relationship to a dimensional deformation of the flex circuit. The flex circuit may be adapted to deform due a pulsation of blood. The deformation may be a bending, expansion, or contraction of the flex circuit. The flex circuit may be configured to change in response to an impedance of a surrounding media. The surrounding media may be composed of blood clot and liquid blood. The response to the impedance of the surrounding media may be predictable. The flex circuit may be configured to generate a signal indicative an electrical property indicative of the penetration of liquid into the aneurism sac. The antenna may be a printed inverted F antenna (PIFA) with a meandering line optimized to work at 433, 868, and 2400 MHz. The flex circuit may include a coil forming a variable inductor. The antenna's resonant frequency may vary based on a distance between at least two points around the coil. The coil may be configured to be energized by a magnetic field directed at the coil from outside the patient's body.

Another aspect of the technology includes a system comprising: an antenna configured to be implanted in a region a living body, wherein a frequency response and/or attenuation response of the antenna shifts in response to changes in a physiological condition of the living body at the region; an input/output module outside the living body and configured to actuate the antenna to generate a response signal and to receive the response signal, wherein the response signal indicates a frequency response and/or attenuation response of the antenna; and a processor configured to: detect whether the frequency response and/or attenuation response indicates a shift in the frequency and/or attenuation response of the antenna; and in response to the detection of the shift issue and/or record a notice that a change occurred in the physiological condition at the region inside the living body.

The system may further include a stent graft configured to be implanted in the region of the living body. The antenna may be mounted to the stent graft. The physiological condition may be an endoleak. The region may be an isolated aneurism sac.

The physiological condition may be a process of blood clotting happening over time. Optionally, the process can be naturally reversed by blood leaks into the sac region. Optionally, the reverse process may accelerate and the acceleration may be detected and indicated. Optionally, the region may be the predetermined location inside the sac. Optionally, the predetermined location may be near the distal seal of the EVAR graft. Optionally, the predetermined location may be proximate to a collateral blood vessel terminating in the sac.

The shift may be a shift of a resonant frequency of the antenna. The shift may be an amplitude of the attenuation response. The shift may occur in response to a healing of a wound or surgical incision at the region. The healing may occur in a period seven to thirty days after the antenna is inserted into the region. The processor may be configured to determine whether the shift occurs after the period. The processor may be configured to detect a rate of change of the shift. The processor may be configured to repeatedly detect the shift, store each of the detected shifts and a time of each detected shift, and determine whether to issue and/or record the notice based on the detected shifts. The processor may be configured to detect a change in a resonant frequency of the antenna. The physiological condition may be bleeding at the region. The physiological condition may be an accumulation of body fluid in the region. The antenna may be a stent antenna, and the region may be in a vascular system of the living body. The antenna may be passive and may not be wired to a source of electromagnetic energy. The antenna may be included in a radio-frequency identification (RFID) device implanted in the living body. The RFID device may be a passive RFID device. The RFID device may include a meandered PIFA antenna.

Another aspect of the technology includes a system configured to monitor a physiological condition at a region located inside a living body, the system comprising: an antenna configured to be implanted at the region inside the living body; a signal detector located outside the living body and configured to actuate the antenna to generate a response signal, the signal detector being further configured to receive the response signal; and a processor configured to analyze a trend in or pattern of resonant frequencies and/or attenuations of response signals recorded over a period of time, wherein the processor is configured to activate an indicator and/or record a notice when the trend or pattern deviates from a predicted trend in or pattern of resonant frequencies and/or attenuations of the response signals, and wherein a variation from the predicted trend or pattern is indicative of a change in the physiological condition.

Another aspect of the technology includes a vascular implant system comprising: an antenna configured to generate a signal with a predetermined resonant frequency in response to receiving a radio signal from a transmitter.

### BRIEF DESCRIPTION OF THE DRAWINGS

FIG. 1 is a schematic illustration of an exemplary endoleak detection system.

FIG. 2 is a schematic illustration of an exemplary control system.

FIG. 3 is an illustration of an exemplary RFID.

FIG. 4 illustrates another RFID.

FIG. 5 shows an exemplary attenuation of return signal.

FIG. 6 shows a flow chart for an exemplary method of implanting and monitoring.

FIGS. 7A and 7B illustrate an exemplary stent graft placed in a patient's aorta.

FIG. 8 is an illustration of an exemplary monitoring device.

FIG. 9 is an illustration of an exemplary sensor system.

FIG. 10 is an illustration of an exemplary monitoring device.

### DETAILED DESCRIPTION

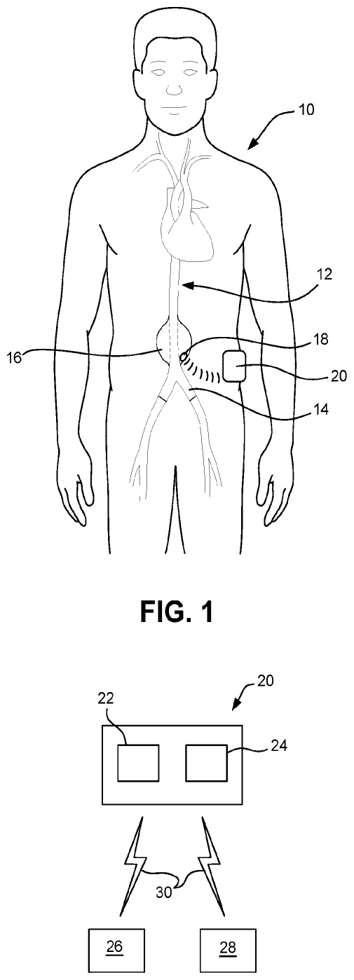

As can be seen in FIG. 1, a patient (or body) 10 has an artery (e.g., aorta) 12 with an aneurism treated with an EVAR stent graft 14 which forms an aneurism sac 16. The sac 16 is equipped with an implantable device 18 that wirelessly communicates with an external console (or reader) 20. The implanted and the external parts of the system can exchange energy and signals through the body 10. The system may be configured to detect early warning of endoleaks while the patient 10 is being monitored at home or in a doctor's office during a periodic checkup visit.

It is contemplated that the reader 20 may be self-contained or may be part of a processing system. As shown in FIG. 2, the reader 20 may include an input/output module 22 and a processor 24. The input/output module 22 may emit energy to the implantable device 18 and may receive a response signal from the implantable device 18. Optionally for active implantable devices 18, the input/output module 22 may receive the signal from the implantable device without first transmitting an actuation signal. In one example, the input/output module 22 may be a radio transmitter and receiver.

The processor 24 may analyze the signal received from the implantable device 18. The processor 24 may also initiate communication between the console 20 and the implantable device 18. In addition, the processor 24 may facilitate communication between a remote system 26 (e.g., remote server or console) or a network 28 by way of data links 30. The data links 30 may be wireless or wires connectable to the remote system 26 and/or network 28.

It is contemplated that the processor 24 and/or the remote system 26 and/or the network 28 may contain an artificial intelligence that may learn from a data trend based on the signals transmitted by the implantable device 18 and may adjust criteria for determining an endoleak based on the patient's healing pattern. A normal healing trend may be anticipated and reversion or deviation from the trend can be interpreted as an alarming event.

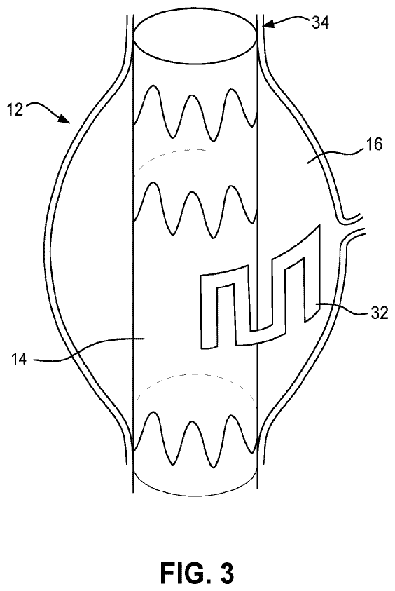

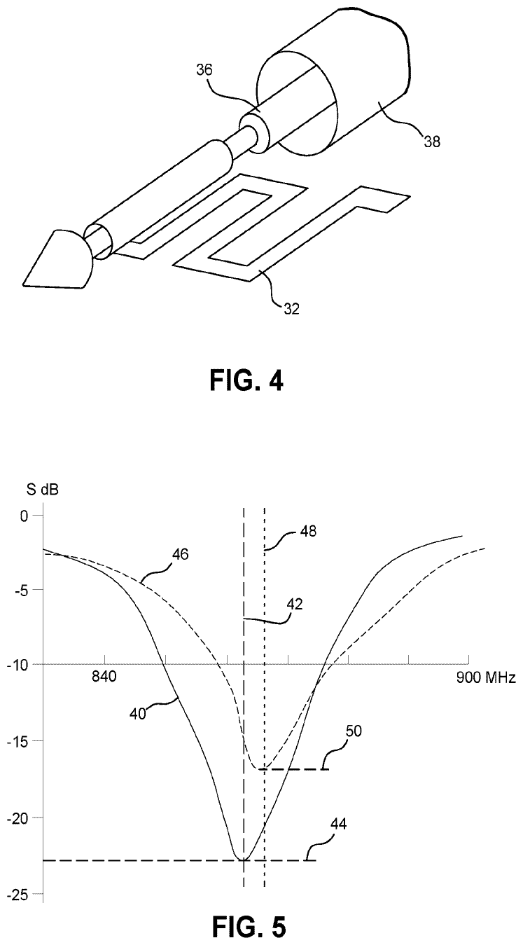

As shown in FIGS. 3 and 4, the implantable device 18 may be in the form of a radio frequency identification (RFID) chip (or antenna or tag) 32. The RFID antenna 32 may be implanted in the aneurism exclusion sac 16 of the artery 12. In addition, the RFID antenna 32 may include a biocompatible dielectric polymer substrate and superstrate. The antenna may be a coil antenna and an encapsulated coil antenna. It is appreciated that many topologies are available for an antenna designer to choose from that can be in some embodiments adapted for delivery through a catheter during the aneurism repair procedure in a collapsed or condensed state.

The RFID antenna 32 can be attached to an artery wall 34 or form an integral part of the stent graft 14 or be inserted into the aneurism sac 16 before the implantation of the stent graft 14. In addition, the RFID antenna 32 may form a tubular shape wrapped around the graft 14. Also, the RFID antenna 32 may be a part of an aneurism sac liner designed to prevent endoleaks. It is contemplated that the implantable device 18 may include several RFID antennas 32 deployed in the same aneurism sac 16.

For illustration purposes the example shows the geometry of the meandered PIFA antenna, which is an example of an RFID antenna. The radiating element may be covered by a bio-compatible superstate and superstrate of dielectric constant of approximately 10 and thickness of 0.1-1.25 mm.

Several RFID antenna designs, such as a miniaturized meandered Planar Inverted-F antenna (PIFA), may be used as the implantable device 18. The RFID antenna 32 may be a printed inverted-F antenna (PIFA) with meandering line and meandering shorting strip under 2.4 GHz. For example, a meander-line PIFA may be optimized to work at 433, 868 and 2400 MHz. These designs are intended as an example only and it is appreciated that there are many designs of RFID antennas 32 that may be used. Flexible, collapsible, and biocompatible designs are available in the right dimensions for possible implantation through a catheter.

Miniaturization may be desired for implantation but may result in a less efficient antenna. In order to avoid the efficiency issues associated with smaller antennae, larger RFID antennas 32 that are flexible and/or foldable may be used. This may allow for the insertion of the RFID antenna 32 into the aneurism sac 16, while increasing the effective area of the RFID antenna 32.

FIG. 4 illustrates a flexible RFID antenna 32 being unrolled from a “flagpole” delivery catheter 36 after the retraction of a sheath 38, thereby greatly increasing its effective area. The RFID antenna 32 may be placed in a specific location where endoleaks may be expected or cover or line a part of the aneurism sac 16 longitudinally or circumferentially. Prior to deployment, the RFID antenna 32 may be tightly rolled into the delivery catheter 36. It is contemplated that the RFID antenna 32 may be folded prior to delivery and then unfolded rather than being rolled up and then unrolled.

Although antennas have been widely used in radio frequency systems, the antenna efficiency, radiation pattern and input impedance may suffer when surrounded in an environment susceptible to loss, such as the human body. Such losses are not constant in the setting of blood leaks (electric shunts). Accordingly, the radio propagation between the RFID antenna 32 and the reader 20 may be used to characterize the tissue along the propagation pathway. This observation, traditionally considered a nuisance in the design and use of RFID antennas, may be exploited.

This configuration may be considered a “sensor-less” system because there is no difference from an operative and structural point of view, between the antenna and a sensor. More precisely, the antenna is the sensor, and the sensor is the antenna. The sensitivity and selectivity of the system are thus strictly connected to the antenna's feature, in particular to its quality factor, and to its bandwidth.

The “sensor-less” configuration discussed above takes advantage of the effect in which the return loss of an optimized RFID antenna 32 will depend on the surrounding tissues and if these tissue properties change, so will the loss. Impedance matching characteristics may be integrated into the design of antennas at popular 433, 868 MHz and 2400 MHz resonant frequencies, for example, as well as other less commonly used frequencies. It is also contemplated that most body tissues are highly conductive (and, therefore, very susceptible to loss) at higher frequencies and that blood clots may have lower resistance and higher capacitance than blood, thereby greatly increasing the losses encountered in the communication with an RFID antenna 32 implanted in the aneurism sac 16 when the leak occurs.

Further exploiting the same principle, antenna's resonant frequency detuning and impedance mismatch may be useful in determining leaks. An antenna tuned to optimally resonate in the “dry” blood clot in the sac may experience a shift away from the resonant frequency when the blood leak is present. A small change in the resonant frequency of the implant, even as small as a shift from 868 to 858 or 878 MHz is observable and detectable by the extracorporeal analysis. For example, it has been measured that for 686 MHz, the antenna relative permittivity of fat is 10 times higher than that for muscle. The contrast between blood and a blood clot may be even more significant because blood is extremely conductive compared to tissues. Thus, changes in the return loss measured in dB down attenuation may indicate changes in the dielectric properties of the aneurism sac. Periodic assessment of these losses may be used to detect clinically significant endoleaks.

FIG. 5 shows how a return signal profile from an exemplary RFID antenna 32 may change when there is an endoleak into the aneurism sac 16. The signal profile 40 is a return signal from an RFID antenna 32 when there is no or very little leakage into the aneurism sac 16. As can be seen, the resonant frequency 42 of the signal profile 40 is 863 MHz and the return loss 44 of the signal at the resonant frequency 42 is −23 dB.

The return signal profile 46 is a return signal from an RFID antenna 32 when there is an endoleak in the aneurism sac 16. As can be seen, the resonant frequency 48 has shifted to 868 MHz. In addition, because the blood leaking into the aneurism sac 16 is more conductive than tissue, the return loss 50 of the signal at the resonant frequency 48 is less (i.e., −17 dB).

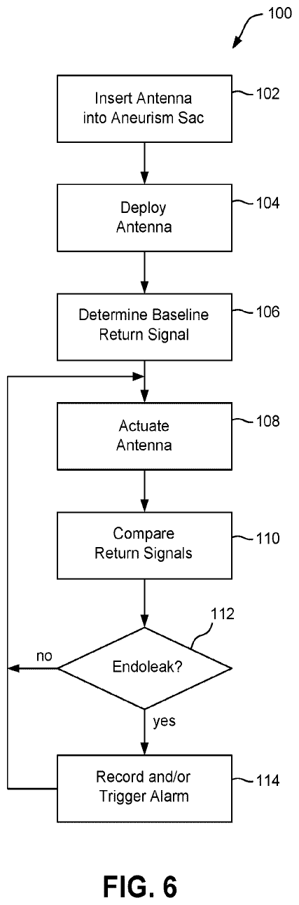

FIG. 6 illustrates a method 100 for implanting an RFID antenna 32 and monitoring a stent graft for an endoleak. The method 100 may begin at step 102 in which the RFID 32 is implanted into the aneurism sac 16. This step may be performed at the same time as the aneurism repair is performed by the surgeon or immediately following the stent graft implantation procedure using the same vascular access techniques, imaging and access devices. As disclosed above, an RFID antenna 32 may be rolled or folded up inside a catheter 36 and then unfurled once inside the aneurism sac 16 (step 104). In the collapsed configuration, the RFID 32 may have an overall diameter sufficient to fit in the catheter, such as 14 F (French) to 17 F. In the expanded, deployed condition, the RFID antenna 32 may have an overall diameter or circumference sufficient to securely engage the artery wall lumen at the anchoring location.

The RFID antenna 32 may include anchor elements to secure and immobilize the antenna in the aneurism sac 16. In addition, it is contemplated that the RFID antenna 32 may be integrally formed within the stent wall or attached to the wall or struts of the stent.

Once the RFID antenna 32 is deployed and secured within the aneurism sac 16, a baseline return signal from the RFID antenna 32 may be triggered and recorded (step 106). It is contemplated that a predetermined amount of time may be allowed to pass between the performance of step 104 and step 106. For example, the user may wait one day to up to a week after the RFID antenna 32 has been implanted. Waiting a predetermined amount of time before triggering the RFID antenna 32 may allow some degree of healing to occur from the stent graft procedure in order to get a more accurate base line reading.

Once patient is ready for the initial triggering of the RFID antenna 32, the reader 20 may be positioned against the patient's skin or clothing, and a radio signal may be emitted from the reader 20. The radio signal from the reader 20 will trigger a response from the RFID antenna 32. The response will be in the form of a unique signal profile that will form a base signal to which future signal profiles will be compared.

It is appreciated that when the electric or acoustic impedance is concerned, pathways from the skin surface to the aortic aneurism sac is posterior or posterior lateral where the tissues are reasonably consistent and represent layers of skin, fat and muscle. Anterior approaches involve highly variable intestines and generally longer distance from the skin to the aorta. Left flank posterior latera vector may be preferred to avoid interference from the vena cava.

After a predetermined amount of time has passed since the initial reading of the RFID antenna signal (step 106), a follow up reading may be performed (step 108). It is contemplated that the amount of time between readings may be one hour, one month, six months, one year, or any time in between. The follow up readings may be periodic at regular intervals. In addition, each reading should be taken from the same or close to the same location to minimize discrepancies caused by the patient's anatomy.

Once the subsequent reading has been performed, the processor 24 of the reader 20 may analyze the return signal from the RFID antenna 32 by comparing the return signal to previous return signals and/or the initial return signal (step 110). It is contemplated that the processor 24 and/or the remote system 26 and/or the network 28 may contain an artificial intelligence that may learn from a data trend based on the signals transmitted by the RFID 32 and may adjust criteria for determining an endoleak based on the patient's healing pattern and/or other factors (e.g., change in weight or body composition). A normal healing trend may be anticipated and reversion or deviation from the trend can be interpreted as an endoleak (step 112). If the processor 24 determines that there is no endoleak or that the volume of the endoleak is below a threshold level, the processor 24 may record the signal and perform step 108 at a later time or date. The schedule of checks can be adjusted based on the clinical risks for the individual patents or the dynamic of the trends of investigated parameters. For example, in a high risk patient the checks may be made more frequently and information may be flagged for the physician monitoring data remotely using the network connection. The step 112 can include such automated adjustments to the frequency of monitoring.

However, if the processor 24 determines that a leak has occurred, the processor 24 may record the return signal and trigger an alarm or notification receivable by a user (step 114). Depending on the severity of the endoleak, the processor 24 may merely record the return signal and perform step 108 again at a later date. Alternatively, the processor 24 may only trigger the alarm and/or notification. Regardless of the response of processor 24 in step 114, step 108 may be performed again at a later date unless the monitoring procedure has been terminated.

It is contemplated that endoleaks may be determined by analyzing a trend in or pattern of resonant frequencies and/or attenuations in response signals recorded over a period of time. The reader 20 may record multiple response signals over a period of time, which may begin after the antenna 32 is implanted. The period of time may be hours, one month, one year, or any time in between.

The processor 24 may analyze the trend in or pattern of resonant frequencies and/or attenuations in the response signal and compare the trend to a predicted trend or pattern that is indicative of an aneurism sac without an endoleak. Any deviation from the predicted trend or pattern may indicate the presence of an endoleak.

RFID tags can be categorized as either passive or active. Passive tags operate without batteries and store information in read-only form. Being battery-less, RFID tags offer longer lifetime. Active RFID tags contain an internal battery and can transmit data over longer range. The RFID antenna system disclosed above may utilize passive RFID antennae. Alternatively, the RIFID antennae may be active.

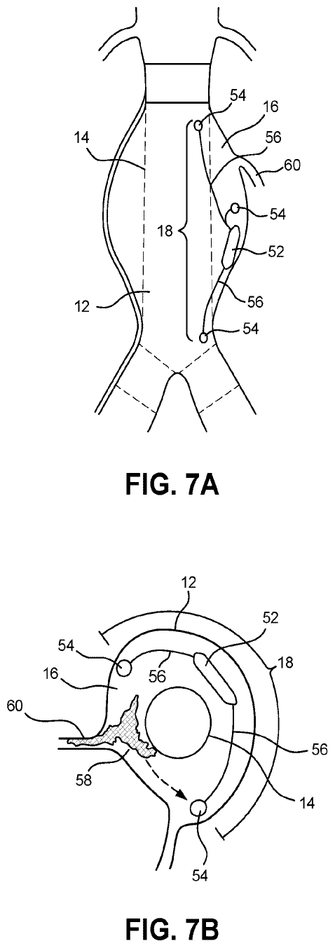

FIGS. 7A and 7B illustrate another system for monitoring endoleaks. In this configuration, the stent graft 14 is placed in the patient's artery 12 to exclude the aneurism sac 16. The implantable device 18 may include a monitoring device 52 and connected remote electrodes 54 implanted in the sac 16. The monitoring device 52 may be connected to the electrodes 54 by way of isolated conductors 56 that may be strategically placed inside the sac 16 to create current pathways where leaks can be expected. Blood 58 is shown entering the sac 16 from the artery 60 that may create an electric current shunt between the electrodes 54 or the monitoring device 52 itself, which may comprise a current return electrode. Electrodes 54 may include accelerometers in addition or as an alternative to impedance sensors. It is expected that accelerometers will become embedded and encapsulated in the sac 16 after clotting but still sensitive to blood pulsations conducted by an elastic clot. Healthy clotting of the sac 16 may attenuate the amplitude of blood pulsations by 50% or more. If an increase of pulsations is detected it can be attributed to endoleaks. For Type II endoleaks, the pressure pulse profile can be expected to be different from the aortic pressure pulse. Sensitive accelerometers may detect retrograde blood flow by analyzing pressure profile for the signs of retrograde collateral flow into the sac 16 and incorporate analysis of circulatory delay.

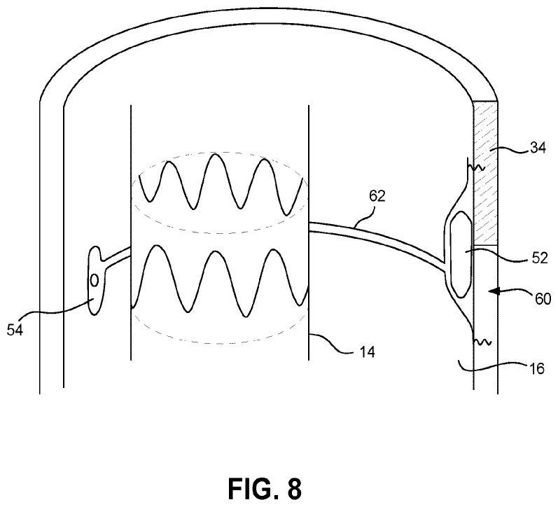

FIG. 8 illustrates the placement of the monitoring device 52 with a remote sensor or electrode 54 in the excluded aneurism sac 16. The electrode 54 may be placed proximate to the artery wall 34 and the proximal edge of the endograft on the side opposite to the monitoring device 52. The monitoring device 52 may be affixed to the wall with barbs or screws or braced inside the sac 16 to mitigate migration. It is anticipated that the electrode 54 will eventually become covered by endothelium or other living tissues typical at the site of the healed injury.

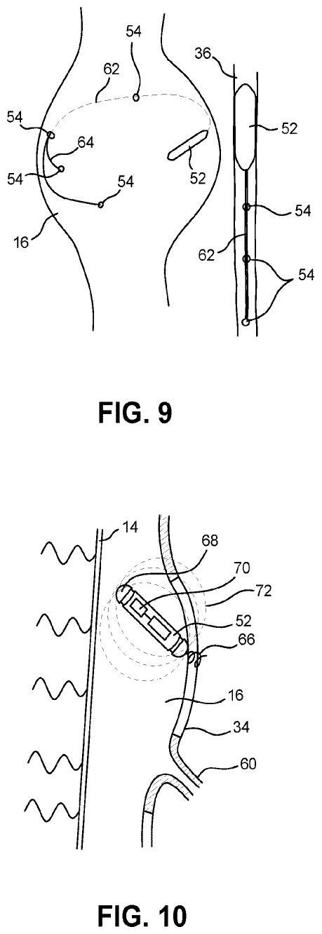

FIG. 9 illustrates the deployment of a system that includes multiple electrodes 54 distributed along the resilient support 62 that can be made of a nitinol shape memory alloy and incorporate insulated wires for delivery of energy, such as excitation signals, and sensing of current. In the delivery state the device may be placed inside a tubular delivery catheter 36. When pushed out of the catheter and deployed inside the aneurism sac 16, the device assumes a helical shape that will circle the aneurism and distribute electrodes 54 at the points that create current pathways likely to detect blood shunts. The support structure may form side branches (branching electrodes) 64 to cover current pathways in the area of interest where endoleaks may be expected.

There is an advantage to the device being a completely self-contained capsule with an internal battery. In one embodiment, such injectable capsule can comprise electrodes measuring impedance on two opposite sides, electronic PCB assembly and an accelerometer encapsulated inside. It may be equipped with a retention mechanism to anchor it to the wall of the aneurism sac 16 in the desired location where the endoleak is likely. A very small, encapsulated battery can last several years since the device will mostly sleep and may only wake up for a couple of minutes every day or night to make measurements and store them in memory. Once in a while the device may use an RF link to transmit accumulated data to an external device.

FIG. 10 illustrates the self-contained monitoring device 52 that may be injected into the aneurism sac 16 using a catheter or a sheath and attached to the artery wall 34 with an anchor screw 66 proximate to an artery 60. Inside the device are battery and electronics that may include an RF antenna. Electrode 68 and the opposing electrode that can be an anchor 70 that create the electric field 72 that forms a current return pathway for the measurement of electric impedance in the sac 16.

It is also appreciated that wireless radio waves or single use batteries waves may not be an ideal form of energy delivery because of relatively high cost of such devices. Alternative ways of harvesting mechanical energy from vibrations have been proposed in the past to power implanted monitors.

Since the abdominal aorta can be located deep inside the body, RF energy transmission may be significantly attenuated. As an alternative, ultrasound electrical recharging may be used. Ultrasound electrical recharging takes advantage of the physics of the body that is mostly water and highly permeable to ultrasound waves at frequencies>20 KHz.

Ultrasound can be used to recharge batteries or capacitors or to provide power directly to a device. Capacitors have advantage over batteries in implanted devices since they do not contain toxic chemicals.

It is appreciated that the electric circuit needed to measure bio-impedance is simpler, less influenced by noise and less power demanding than the circuit needed to monitor hydraulic pressure, especially absolute pressure. It is also appreciated that the vibration frequencies used by ultrasound energy transmission are in the range of electric waveforms that can be used to test local electric impedance of tissues. These piezoelectric vibrations may be directly converted into electric waveform applied to tissues inside the aneurism sac 16 to detect a drop in impedance that may be created by blood shunting.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both, unless the this application states otherwise. Furthermore, characteristics or steps which have been described may also be used in combination with other characteristics or steps and in any order unless the disclosure or context suggests otherwise.

### FIGURES AND ELEMENTS

10 Patient
12 Artery
14 Stent Graft
16 Aneurism sac
18 Implantable device
20 Console/reader
22 Input/output module
24 Processor
26 Remote system
28 Network
30 Data links
32 RFID antenna
34 Artery wall
36 Catheter
38 Sheath
40 Signal Profile
42 Resonant frequency
44 Return loss
46 Signal profile
48 Resonant frequency
50 Return loss
52 Monitoring device
54 Sensor Electrode
56 Conductors
58 Blood
60 Artery
62 Resilient support
64 Branching electrodes
66 Fixation screw
68 Electrode
70 Anchor
72 Electric field
100 Method
Step 102
Step 104
Step 106
Step 108
Step 110
Step 112
Step 114

1. A system configured to monitor a physiological condition at a region located inside a living body, the system comprising:
an antenna configured to be implanted at the region inside the living body;
a signal detector located outside the living body and configured to actuate the antenna to generate a response signal, the signal detector being further configured to receive the response signal; and
a processor configured to compare a resonant frequency and/or an attenuation of the response signal to a reference,
wherein the processor is configured to activate an indicator and/or record a notice when the resonant frequency and/or the attenuation varies from the reference, and
wherein a variation from the reference is indicative of a change in the physiological condition.

2. The system of claim 1, wherein the reference is a resonant frequency and/or attenuation of a previously recorded response signal.

3. The system of claim 1, wherein the reference is a calculated trend in or pattern of resonant frequencies and/or attenuations of previous response signals recorded over a predetermined period of time.

4. The system of claim 3, wherein the predetermined period of time begins after the antenna has been implanted in the region.

5. The system of claim 1, wherein the region is an aneurism sac.

6. The system of claim 1, wherein the physiological condition is an amount of blood or fluid leaking into the aneurism sac.

7. The system of claim 1, wherein a difference between the resonant frequency of the response signal and the resonant frequency of the reference signal that is greater than a threshold indicates a leakage of blood into the aneurism sac.

8. The system of claim 1, wherein a difference between the attenuation of the response signal and the attenuation of the reference signal that is greater than a threshold indicates a leakage of blood into the aneurism sac.

9. The system of claim 1, wherein the antenna is periodically actuated, and the characteristic of the subsequent response signal is compared to the characteristic of the reference signal and/or previous response signals.

10. The system of claim 1, wherein the antenna is a radio-frequency identification (RFID) device.

11. The system of claim 1, wherein the signal detector is configured to receive the response signal on a posterior side of the living body.

12. A system for monitoring a physiological condition inside a patient's body, the system comprising:
a passive implantable antenna adapted for placement in an aneurism sac of the patient during an endovascular procedure; and
a transmitter and a receiver that are external to the patient's body,
wherein the transmitter is configured to trigger a response signal from the passive implantable antenna, and
wherein the receiver is configured to determine the presence of an endoleak in the aneurism sac based on a shift in the antenna's resonant frequency and/or the antenna's impedance.

13. The system of claim 12, wherein the transmitter and the receiver are configured to be in radio communication with the antenna.

14. The system of claim 12, wherein the receiver is configured to receive a radio signal indicative of the antenna's resonant frequency and/or impedance, and wherein the shift of the resonant frequency and/or or a change in the impedance is indicative of a penetration of liquid into the aneurism sac.

15. The system of claim 12, wherein the receiver is configured to receive a radio signal indicative of the antenna's resonant frequency and/or impedance, and wherein a deviation from a trend in or pattern of resonant frequencies and/or impedance of the response signals is indicative of a p

16. The system of claim 12, wherein the antenna is configured so that the resonant frequency varies based on the capacitance of the surrounding environment, and the variance of the resonant frequency is indicative of a difference between a clot and liquid blood.

17. The system of claim 12, wherein the receiver is configured to determine the presence of an endoleak by detecting a gradual increase of signal loss encountered when communicating with an RFID antenna.

18. The system of claim 12, wherein the receiver is configured to determine a catastrophic endoleak by detecting an abrupt increase in signal loss encountered when communicating with an RFID antenna.

19.-56. (canceled)
